(19) United States
(12) Patent Application Publication
(10) Pub. No.: US 2015/0265227 A1
(43) Pub. Date: Sep. 24, 2015

(54) X-RAY APPARATUS

(71) Applicant: SHIMADZU CORPORATION, Kyoto (JP)

(72) Inventors: Satoshi SANO, Kyoto (JP); Toshiyuki SATO, Kyoto (JP); Koichi TANABE, Kyoto (JP); Toshinori YOSHIMUTA, Kyoto (JP); Hiroyuki KISHIHARA, Kyoto (JP); Takahiro DOKI, Kyoto (JP)

(21) Appl. No.: 14/663,240

(22) Filed: Mar. 19, 2015

(30) Foreign Application Priority Data
Mar. 20, 2014 (JP) 2014-058747

(51) Int. Cl. A61B 6/00

(52) U.S. Cl. CPC A61B 6/4042; A61B 6/4233; A61B 6/542

(57) ABSTRACT

Disclosed is an X-ray apparatus with an X-ray tube controller. The X-ray tube controller controls an X-ray tube so as for X-rays emitted from the X-ray tube to have an energy width whose upper limit is more than the minimum K-shell absorption edge of K-shell absorption edges for elements forming a conversion film and is equal to or less than a preset value depending on a K-shell absorption edge corresponding to a characteristic X-ray whose energy influences the blur. Accordingly, the less number of ejected K-shell characteristic X-rays is obtainable than the case when the emitted X-rays have an energy width whose upper limit is more than a preset value depending on the K-shell absorption edge corresponding to the characteristic X-ray whose energy influences the blur. This allows a suppressed blurred image generated from ejected K-shell characteristic X-rays outside a pixel area where X-rays enter to introduce a photoelectric effect.

### CROSS-REFERENCE TO RELATED APPLICATIONS

This application claims priority to Japanese Patent Application No. 2014-058747 filed Mar. 20, 2014 the subject matter of which is incorporated herein by reference in entirety.

### TECHNICAL FIELD

The present invention relates to an X-ray apparatus conducting X-ray radiography by emitting X-rays to a subject and detecting X-rays passing through the subject.

### BACKGROUND ART

A currently-used X-ray apparatus includes an X-ray tube that emits X-rays to a subject, and an X-ray detector detecting X-rays passing through the subject. See, for example, Japanese Patent Publication No. 2013-019698A.

The X-ray detector is classified by two types in terms of detecting X-rays. That is, the two types are indirect conversion and direct conversion types. In the indirect conversion-type X-ray detector, X-rays are converted into another type of light with scintillators, and then the light is converted into electric charges (electron-hole pairs) with a photodiode or a CCD image sensor, whereby X-rays are detected. In contrast to this, in the direct conversion-type X-ray detector, incident X-rays are converted into electric charges with a semiconductor film, whereby X-rays are detected.

With the indirect conversion-type detector, an X-ray reaction position of the scintillator differs from a position where a photodiode catches X-rays. In contrast to this, with the direct conversion-type detector, electric charges (electrons or holes) drift from an X-ray reaction position to collecting electrodes in the semiconductor film directly. Consequently, the direct conversion-type detector achieves a positional resolution superior to that of the indirect conversion-type detector. Examples of the currently-used direct conversion-type detector include a semiconductor device composed of Si (silicon), CdTe (cadmium telluride), CdZnTe (Cadmium zinc telluride), PbI₂ (lead iodide), TlBr (thallium bromide), and the like.

Moreover, the X-ray detector is classified by two reading systems, i.e., an integral reading system and a photon counting system. In the integral reading system, the converted electric charges are stored in a storage capacitor for a given period of time, and thereafter the stored electric charges are read out with switching elements such as TFTs (thin-film transistor). In contrast to this, in the photon counting system, one photon of X-rays is counted at a time.

Moreover, examples of the X-ray detector include one with fine pixels of 10 μm level. Such an X-ray detector is formed with an SOI (Silicon-On Insulator) technology.

### SUMMARY OF INVENTION

### Technical Problem

The direct conversion type X-ray detector has a higher resolution than the indirect conversion-type X-ray detector. However, as pitches of the pixel electrodes (pixel pitches) become smaller, an image to be captured contains a blur due to the characteristic X-ray generated upon photoelectrical conversion.

The above is to be described in detail. When X-rays enter into a conversion film to introduce photoelectrical conversion, the characteristic X-ray is ejected. An element with a larger atomic number has a high ejection probability of characteristic X-rays. When the conversion film is composed of CdTe, a K-shell characteristic X-ray of approximately 30 keV is ejected. When the K-shell characteristic X-ray is ejected out of a pixel area where photoelectrical conversion occurs, an electric charge may be generated in another pixel area. Here, such a phenomenon as the K-shell characteristic X-ray is ejected out of the pixel area where photoelectrical conversion occurs is referred to as “K-escape” hereinunder as appropriate. The K-shell characteristic X-ray of 30 keV has an attenuation length of approximately 100 μm in the conversion film of CdTe. A finer pixel (pixel electrode) causes a larger blur in an image due to the K-shell characteristic X-rays.

The photon counting method is also adopted for a measure against the blur in the image. In this method, the number of electric charges (pulse height values) corresponding to the K-shell characteristic X-ray is cut-off from the number of electric charges converted with a conversion film upon incidence of X-rays into the conversion film at a preset threshold. In this manner, the electric charges corresponding to the K-shell characteristic X-ray are removed. However, with the method, almost the number of electric charges converted with the conversion film is cut-off. This causes a large wasted dose of X-rays. As a result, a more dose of X-rays are needed for generating an image.

The present invention has been made regarding the state of the art noted above, and its one object is to provide an X-ray apparatus that obtains an image with a suppressed blur due to a characteristic X-ray generated upon photoelectrical conversion. In addition, another object is to provide an X-ray apparatus that allows suppression in wasted dose of X-rays incident into a conversion film.

### Solution to Problem

The present invention is constituted as stated below to achieve the above object. One embodiment of the present invention discloses an X-ray apparatus conducting X-ray radiography. The X-ray apparatus includes an X-ray tube emitting X-rays to a subject; an X-ray detector detecting X-rays passing through the subject; and an X-ray tube controller controlling the X-ray tube. The X-ray detector includes a conversion film and collecting electrodes. The conversion film is composed of many different types of elements, and converts incident X-rays into electric charges. The collecting electrodes are provided on at least one face of the conversion film, and each collect the electric charge converted with the conversion film. The X-ray tube controller controls the X-ray tube so as for the X-rays emitted from the X-ray tube to have an energy width whose upper limit is more than the minimum K-shell absorption edge of K-shell absorption edges for the elements forming the conversion film, and is equal to or less than a preset value depending on a K-shell absorption edge of the K-shell absorption edges for the elements corresponding to a characteristic X-ray whose energy influences a blur.

Another embodiment of the present invention discloses an X-ray apparatus conducting X-ray radiography having a conversion film composed of one type of element. That is, disclosed is an X-ray apparatus conducting X-ray radiography including an X-ray tube emitting X-rays to a subject; an X-ray detector detecting X-rays passing through the subject; and an X-ray tube controller controlling the X-ray tube. The X-ray detector includes a conversion film and collecting electrodes. The conversion film is composed of one type of element and converts incident X-rays into electric charges. The collecting electrodes are provided on at least one face of the conversion film, and each collect the electric charges converted with the conversion film. The X-ray tube controller controls the X-ray tube so as for the X-rays emitted from the X-ray tube to have an energy width whose upper limit is more than a K-shell absorption edge for the element, and is equal to or less than a preset value depending on the K-shell absorption edge for the element corresponding to the characteristic X-ray whose energy influences the blur.

With the X-ray apparatus according to the present embodiment, the X-ray tube controller controls the X-ray tube so as for the X-rays emitted from the X-ray tube to have an energy width whose upper limit is more than the minimum K-shell absorption edge of K-shell absorption edges for the elements forming the conversion film and is equal to or less than a preset value depending on a K-shell absorption edge of the K-shell absorption edges for the elements corresponding to a characteristic X-ray whose energy influences a blur. Moreover, the X-ray tube controller controls the X-ray tube so as for the X-rays emitted from the X-ray tube to have an energy width whose upper limit is more than a K-shell absorption edge for the element corresponding to a characteristic X-ray whose energy influences a blur, and is equal to or less than a preset value depending on the K-shell absorption edge for the element corresponding to the characteristic X-ray whose energy influences the blur. That is, the X-ray tube controller controls the upper limit of the energy width of emitted X-rays depending on the K-shell absorption edge of the elements that form the conversion film. Accordingly, the less number of ejected K-shell characteristic X-rays is obtainable than the case when the emitted X-rays have an energy width whose upper limit is more than a preset value depending on the K-shell absorption edge for the element or the K-shell absorption edge of the K-shell absorption edges for the elements corresponding to the characteristic X-ray whose energy influences the blur. Consequently, a suppressed blur in an image is obtainable, the blur occurring due to ejection of the K-shell characteristic X-ray outside a pixel area where X-rays enter to introduce a photoelectric effect.

Moreover, the X-ray detector of the X-ray apparatus further includes a charge-voltage converter, a comparator, and a collecting device. The charge-voltage converter converts the electric charge collected in the collecting electrodes individually into a voltage signal. The comparator outputs a photon detection signal, indicating detection of one photon, if the voltage signal converted with the charge-voltage converter is higher than a preset threshold, at the preset threshold a voltage signal equal to or less than energy corresponding to a K-shell characteristic X-ray is cut off. The collecting device counts the number of photons for each of pixels in accordance with the photon detection signal. Such is preferable.

The comparator contains the threshold preset to cut off a voltage signal corresponding to the energy of K-shell characteristic X-ray whose value is equal to or less than the threshold. If the voltage signal converted with the charge-voltage converter is higher than the preset threshold, the comparator outputs a photon detection signal indicating detection of one photon. Accordingly, suppressed detection of the photon is obtainable within a pixel other than a pixel to which X-rays enter. This leads to a suppressed blur in the image. Moreover, as noted above, control of the X-ray tube controller causes suppressed ejection of a K-shell characteristic X-ray. This causes an abrupt distribution of the voltage signals obtained from the incident X-rays. Consequently, reduction in number of detected photons can be suppressed. The reduction occurs when the comparator discriminates the electric signal in the pixel to which emitted X-rays enter as no photon detection using the preset threshold. That is, suppression in wasted dose of X-rays is obtainable.

Moreover, the K-shell absorption edge corresponding to the characteristic X-ray whose energy influences the blur is preferably 15 keV or more. Specifically, a characteristic X-ray corresponding to the K-shell absorption edge of more than 15 keV influences the blur. On the other hand, if the K-shell absorption edge is less than 15 keV, the K-shell characteristic X-ray has a small attenuation length, and thus insufficiently spreads even upon ejection of K-shell characteristic X-rays. Moreover, energy of the K-shell characteristic X-ray to be ejected is low. Accordingly, an amount of electric charges generated by the K-shell characteristic X-ray is also small. For instance, a K-shell characteristic X-ray ejected from an element Br of TlBr has energy of around 13 keV, and an attenuation length of around 20 μm. In addition, the K-shell characteristic X-ray with the energy of around 13 keV generates a less amount of electric charges.

Moreover, the conversion film of the X-ray apparatus according to the present embodiment is composed of many different types of elements. A K-shell absorption edge among K-shell absorption edges for the elements forming the conversion film, other than the K-shell absorption edge corresponding to the characteristic X-ray whose energy influences the blur, is lower than a K-shell absorption edge for Cd. Such is preferable. This achieves low energy of the K-shell characteristic X-ray to be ejected. Consequently, a less amount of electric charges generated from the K-shell characteristic X-ray is obtainable. Accordingly, a less amount of electric charges is generated even when the K-shell characteristic X-ray reaches the pixel other than the pixel to which incident X-rays enter to introduce a photoelectric effect. This achieves the suppressed blur in the image.

Moreover, in the X-ray apparatus according to the present embodiment, the K-shell absorption edge corresponding to the characteristic X-ray whose energy influences the blur is higher than a K-shell absorption edge for Te. Such is preferable. Accordingly, an upper limit of an energy width of emitted X-rays is set with reference to the K-shell absorption edge. Accordingly, emission of X-rays with higher energy is obtainable.

Moreover, the conversion film of the X-ray apparatus according to the present embodiment is composed of many different types of elements. A K-shell absorption edge among K-shell absorption edges other than the K-shell absorption edge corresponding to the characteristic X-ray whose energy influences the blur has energy with which the K-shell characteristic X-ray to be ejected has an attenuation length smaller than twice a pitch of the collecting electrode. Such is preferable. Accordingly, the K-shell characteristic X-ray to be ejected falls within an area smaller than twice the pitch of each of the collecting electrodes. This achieves the suppressed blur in the image.

Moreover, the X-ray tube controller of the X-ray apparatus according to the present embodiment controls the X-ray tube so as for the upper limit of the energy width to be more than the K-shell absorption edge corresponding to the characteristic X-ray whose energy influences the blur and to be equal to or less than a preset value depending on the K-shell absorption edge corresponding to the characteristic X-ray whose energy influences the blur. Such is preferable. Accordingly, emission of X-rays with much higher energy is obtainable.

Moreover, the collecting electrodes of the X-ray apparatus according to the embodiment each preferably have a pitch equal to or less than several tens μm. Accordingly, the suppressed blur in the image is obtainable when the pitch of the collecting electrode is equal to or less than several tens μm.

### Advantageous Effects of Invention

With the X-ray apparatus according to the present embodiment, the X-ray tube controller controls the X-ray tube so as for the X-rays emitted from the X-ray tube to have an energy width whose upper limit is more than the minimum K-shell absorption edge of K-shell absorption edges for the elements forming the conversion film, and is equal to or less than a preset value depending on a K-shell absorption edge of the K-shell absorption edges for the elements corresponding to the characteristic X-ray whose energy influences the blur. Moreover, the X-ray tube controller controls the X-ray tube so as for the X-rays emitted from the X-ray tube to have an energy width whose upper limit is more than a K-shell absorption edge for the element corresponding to a characteristic X-ray whose energy influences a blur, and is equal to or less than a preset value depending on the K-shell absorption edge for the element corresponding to the characteristic X-ray whose energy influences the blur. That is, the X-ray tube controller controls the upper limit of the energy width of emitted X-rays depending on the K-shell absorption edge of the elements forming the conversion film. Accordingly, the less number of ejected K-shell characteristic X-rays is obtainable than the case when the emitted X-rays have an energy width whose upper limit is more than a preset value depending on the K-shell absorption edge for the element or a K-shell absorption edge of the K-shell absorption edges for the elements corresponding to the characteristic X-ray whose energy influences the blur. Consequently, a suppressed blur in the image is obtainable, the blur occurring due to ejection of the K-shell characteristic X-ray outside a pixel area where X-rays enter to introduce a photoelectric effect.

It is to be understood that both the foregoing general description and the following detailed description are exemplary and explanatory and are intended to provide further explanation of the invention as claimed.

### BRIEF DESCRIPTION OF DRAWINGS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

For the purpose of illustrating the invention, there are shown in the drawings several forms which are presently preferred, it being understood, however, that the invention is not limited to the precise arrangement and instrumentalities shown.

FIG. 1 schematically illustrates an X-ray apparatus according to one embodiment of the present invention.

FIG. 2 is a longitudinal sectional view of a flat panel X-ray detector (FPD) according to the embodiment.

FIG. 3 is a plan view of the flat panel X-ray detector (FPD) according to the embodiment.

FIG. 4 illustrates a conventional relationship between a K-shell absorption edge of a semiconductor film and energy of emitted X-rays (X-ray spectrum).

FIG. 5A illustrates a relationship between a K-shell absorption edge of a semiconductor film of CdTe and energy of emitted X-rays (X-ray spectrum). FIG. 5B illustrates a relationship between a K-shell absorption edge of a semiconductor film of TlBr and energy of emitted X-rays (X-ray spectrum).

FIG. 6A is a distribution of detected electric charges using the CdTe semiconductor film. FIG. 6B is a distribution of detected electric charges using the TlBr semiconductor film.

FIG. 7 is a distribution of detected electric charges obtained through integration of FIGS. 6A and 6B in a vertical direction along a paper plane.

FIG. 8 illustrates a flat panel X-ray detector (FPD) according to another embodiment of the present invention.

FIG. 9A is a distribution of detected electric charges in a semiconductor film of CdTe with no threshold discrimination. FIG. 9B is a distribution of detected electric charges in a semiconductor film of CdTe with threshold discrimination.

FIG. 10A is a distribution of detected electric charges in a semiconductor film of TlBr with no threshold discrimination. FIG. 10B is a distribution of detected electric charges in a semiconductor film of TlBr with threshold discrimination.

FIG. 11 illustrates a flat panel X-ray detector (FPD) according to one modification of the present invention.

### EMBODIMENT 1

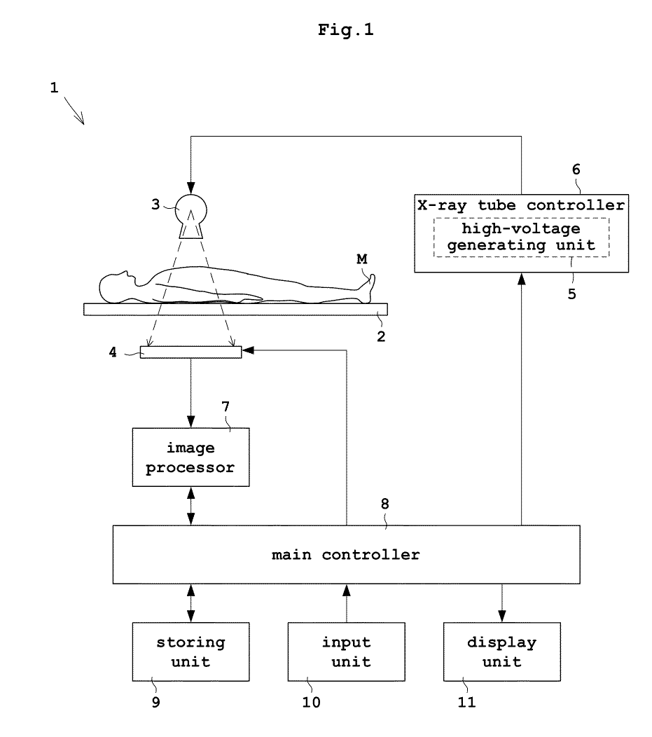

The following describes Embodiment 1 of the present invention with reference to drawings. FIG. 1 schematically illustrates an X-ray apparatus according to Embodiment 1.

<X-Ray Apparatus>

Reference is made to FIG. 1. Firstly, description is made to a construction of the X-ray apparatus 1. The X-ray apparatus 1 includes a top board 2 supporting a subject M placed thereon, an X-ray tube 3 emitting X-rays to the subject M, and a flat panel X-ray detector (FPD: flat panel detector) 4 detecting X-rays passing through the subject M. Hereinunder, the flat panel X-ray detector is referred to as an “FPD” as appropriate. The flat panel X-ray detector (FPD) 4 corresponds to the X-ray detector in the present invention.

The X-ray apparatus 1 further includes an X-ray tube controller 6 with a high-voltage generating unit 5, and an image processor 7. The high-voltage generating unit 5 generates tube voltage and/or tube current of the X-ray tube 3. The X-ray tube controller 6 controls the X-ray tube 3. The image processor 7 performs various processes to an image outputted from the FPD 4. The X-ray tube controller 6 is to be described in detail later.

The X-ray apparatus 1 further includes a main controller 8, a storing unit 9, an input unit 10, and a display unit 11. The main controller 8 controls various components en bloc, such as the X-ray tube 3, the FPD 4, and the X-ray tube controller 6. The storing unit 9 stores the image processed by the image processor 7. The input unit 10 performs input setting by an operator. The display unit 11 displays the image processed by the image processor 7.

The main controller 8 is formed by a central processing unit (CPU) and the like. The storing unit 9 is formed by a storage medium including a demountable one such as a ROM (Read-only Memory), a RAM (Random-Access Memory), and a hard disk. The input unit 10 is formed by a joystick, a mouse, a touch panel, and the like. The display unit 11 is formed by a liquid crystal monitor, and the like.

<Flat Panel X-Ray Detector (FPD)>

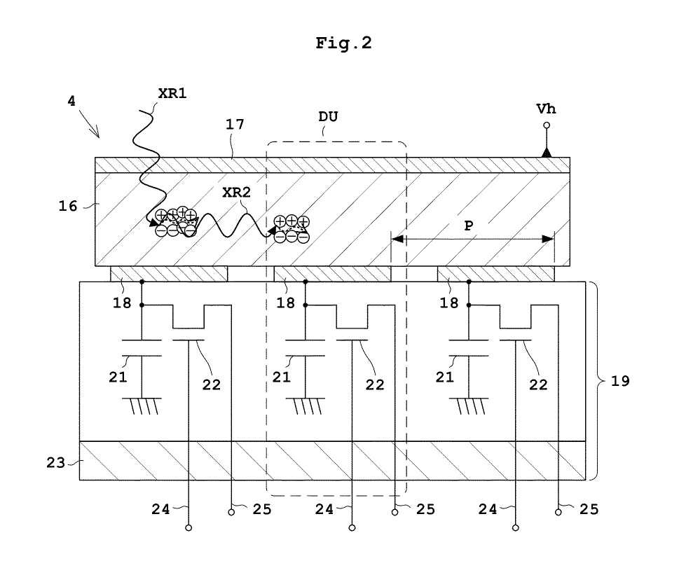

The following describes a construction of the FPD 4. The FPD 4 of the present embodiment is of a storage type. FIG. 2 is a longitudinal sectional view of the FPD 4. In FIG. 2, XR1 denotes an emitted X-ray or an incident X-ray, and XR2 denotes a K-shell characteristic X-ray.

As illustrated in FIG. 2, the FPD 4 includes a semiconductor film 16, a common electrode 17, and pixel electrodes 18. The semiconductor film 16 is sensitive to incident X-rays to generate electric charges. The common electrode 17 is provided on a first face of the semiconductor film 16 for applying bias voltage Vh. The pixel electrodes 18 are arranged on a second face of the semiconductor film 16 in a two-dimensional matrix array. Each of the pixel electrodes 18 has a pitch P of more than 0 and equal to or less than several tens μm (i.e., less than 100 μm). Here, the pixel electrodes 18 correspond to the collecting electrodes in the present invention.

The semiconductor film 16 is composed of one type of element (an element) or many different types of elements. That is, the semiconductor film 16 is composed of Si, Se (selenium), CdTe, CdZnTe, ZnTe (zinc telluride), HgI₂ (mercury iodide), PbI₂, PbO (lead oxide), BiI₃ (bismuth iodide), TlBr, GaAs (gallium arsenide), InP (indium phosphide), and the like. Examples of an element forming the semiconductor film 16 include Si and Se. Examples of two elements forming the semiconductor film 16 include CdTe, ZnTe, PbI₂, PbO, BiI₃, TlBr, GaAs, and InP. Examples of three elements forming the semiconductor film 16 include CdZnTe. Here, four or more elements are applicable as many different types of elements.

The semiconductor film 16 has a film thickness of several hundreds μm or more. This maintains high detection efficiency. Moreover, the pixel electrodes 18, the semiconductor film 16, and the common electrode 17 are formed on an active matrix substrate 19, in this order, through vapor deposition or the like. Here, the semiconductor film 16 corresponds to the conversion film in the present invention.

The active matrix substrate 19 includes capacitors 21, TFTs 22 as switching elements, and an insulating substrate 23. The capacitors 21 each accumulate electric charges generated by the semiconductor film 16. The TFTs 22 each read out the electric charges accumulated in the capacitors 21 individually. The insulating substrate 23 is made of a glass or the like. The capacitors 21, the TFTs 22, the gate lines 24, and data lines 25 are formed on the insulating substrate 23.

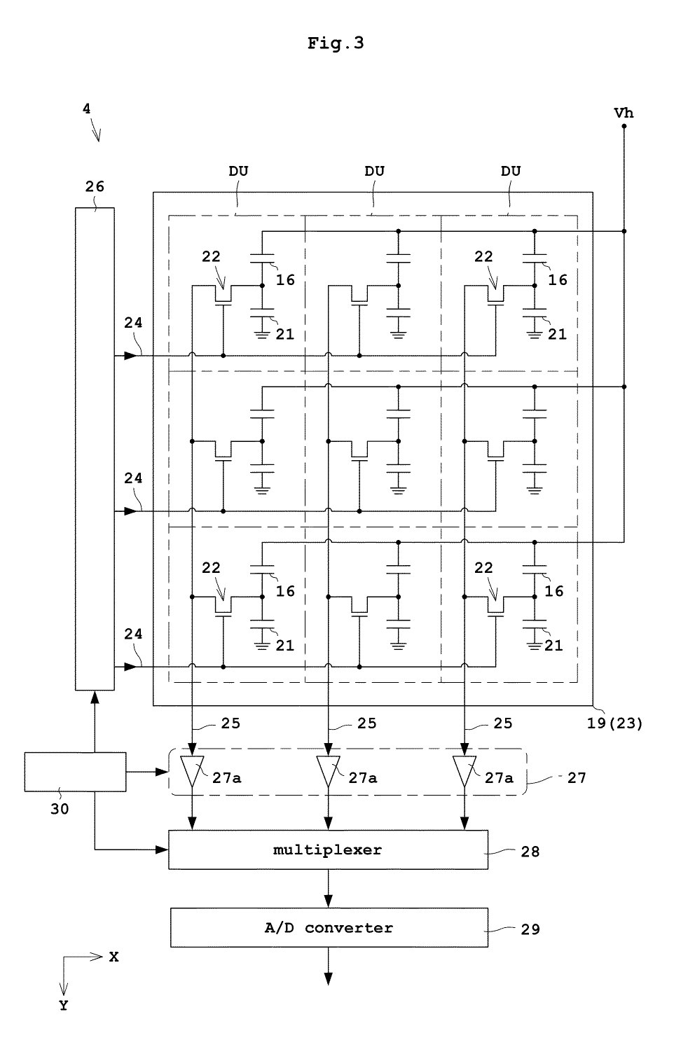

As illustrated in FIG. 2 by dotted lines, an X-ray detecting element DU corresponding to one pixel is formed by the semiconductor film 16, the common electrode 17, a pixel electrode 18, a capacitor 21, and a TFT 22. FIG. 3 is a plan view of the FPD 4. As illustrated in FIG. 3, a plurality of X-ray detecting elements DU is arranged in a two-dimensional matrix. Accordingly, the capacitor 21 and the TFT 22 are provided for each of the pixels in a two-dimensional matrix. Here, FIG. 3 illustrates the X-ray detecting elements DU in 3 by 3 pixels for convenience. For instance, the X-ray detecting elements DU are arranged in 1024 by 1024 pixels.

The active matrix substrate 19 includes the gate lines 24 and the data lines 25. The gate lines 24 each connect a plurality of gates of TFTs 22 arranged in line in a row direction (X-direction) of FIG. 3. The data lines 25 each connect a plurality of sources of TFTs 22 arranged in line in a column direction (Y-direction) of FIG. 3. The capacitor 21 is connected to a drain of the TFT 22.

Moreover, the gate lines 24 are connected to a gate driver circuit 26 at one ends thereof. The gate driver circuit 26 actuates the TFTs 22 in turn for every gate line 24 (for every line). For instance, the gate driver circuit 26 applies drive signals to the gate lines 24 beginning at the top of FIG. 3, thereby turning ON the TFTs 22 connected the gate lines 24. Consequently, the electric charges accumulated in the capacitor 21 are transmitted via the TFTs 22 turned ON to the data lines 25, where the electric charges are read out.

A charge-voltage converter group 27, a multiplexer 28, and an A/D converter 29 are connected in this order to an output side of the data line 25. The charge-voltage converter group 27 amplifies the electric charges to convert the electric charges into voltage signals. The charge-voltage converter group 27 includes amplifiers 27a provided for each of the data lines 25. The multiplexer 28 selects and outputs one of the voltage signals. The A/D converter 29 converts the analog voltage signal into a digital voltage signal.

The gate driver circuit 26, the charge-voltage converter group 27, the multiplexer 28, and the A/D converter 29 are controlled by an FPD controller 30. The FPD controller 30 is controlled by the main controller 8.

<Semiconductor Film and X-Ray Tube Controller>

The following describes one characteristic of the present invention. The X-ray apparatus 1 according to the present embodiment achieves suppression of the blur in the image due to K-shell characteristic X-rays. In the present embodiment, the blur in the image due to the characteristic X-ray is suppressed through control by the X-ray tube controller 6 depending on the semiconductor film 16, i.e., a relationship between energy of a K-shell absorption edge of the semiconductor film 16 and energy of emitted X-rays controlled by the X-ray tube controller 6.

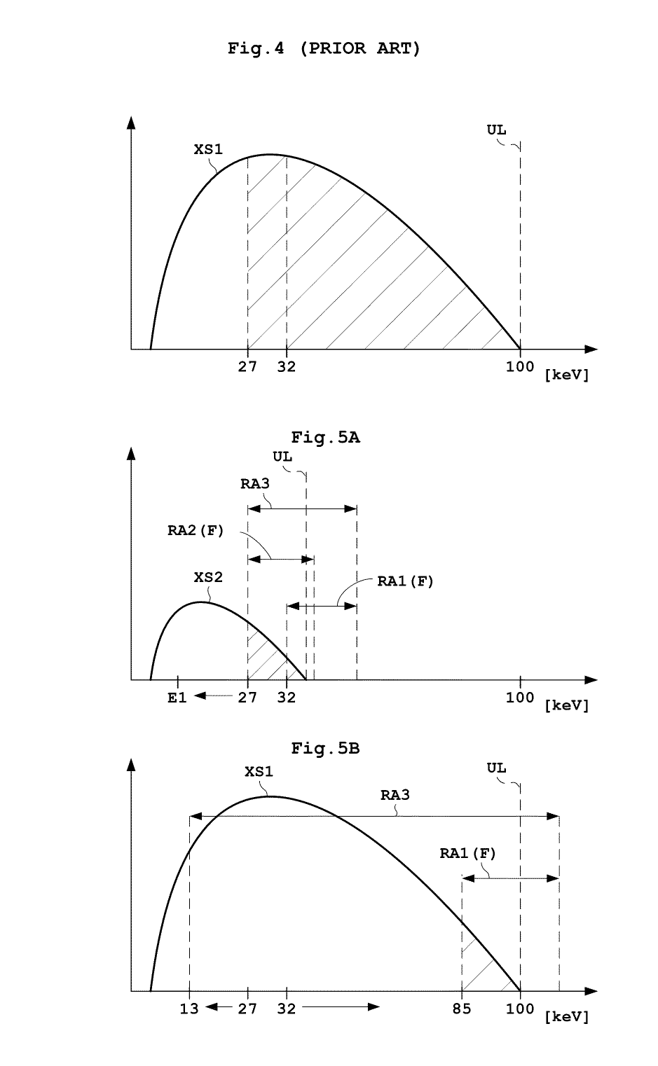

FIG. 4 illustrates a conventional relationship between the K-shell absorption edge of the semiconductor film and energy of emitted X-rays (X-ray spectrum). Here in FIG. 4 as well as FIGS. 5A and 5B to be mentioned later, a horizontal axis indicates energy whose unit is keV, and a longitudinal axis indicates the number (relative number) of X-ray photons.

In FIG. 4, the numeral XS1 denotes an X-ray spectrum of X-rays emitted from the X-ray tube 3 whose set tube voltage is 100 kV. Here, the emitted X-rays have an energy width whose upper limit UL is 100 keV. Moreover, the semiconductor film 16 of CdTe is adopted. A K-shell absorption edge of Cd has energy of approximately 27 keV, whereas a K-shell absorption edge of Te has energy of approximately 32 keV. If X-rays having an energy width whose maximum value (upper limit) is 100 keV are emitted, K-shell characteristic X-rays for Cd and Te are ejected at a probability represented by yield of K-shell characteristic X-rays in Table 1 upon generation of a photoelectric effect. For instance, as illustrated in FIG. 4 by an area with diagonal lines, a K-shell characteristic X-ray is ejected from X-rays whose energy is larger than approximately 27 keV as the K-shell absorption edge for Cd upon the photoelectric effect. Consequently, a large amount of K-shell characteristic X-rays is ejected. In addition, the K-shell characteristic X-rays for Cd and Te are both high, i.e., approximately 30 keV. Moreover, the K-shell characteristic X-rays of approximately 30 keV each have a long attenuation length of approximately 100 μm in CdTe. As a result, a large amount of electric charges are detected within a wide area, causing the blur in the image.

TABLE 1

| | Cd | Te | Tl | Br |
|---|---|---|---|---|
| K-shell | 26.71 keV | 31.81 keV | 85.53 keV | 13.473 keV |
| absorption | | | | |
| edge | | | | |
| K-shell | 0.843 | 0.872 | 0.948 | 0.507 |
| characteristic | | | | |
| X-ray yield | | | | |
| Kα | 23.108 keV | 27.378 keV | 72.167 keV | 11.907 keV |
| Kβ | 26.116 keV | 31.108 keV | 82.4 keV | 13.288 keV |

Then, as illustrated in FIG. 5A, the X-ray tube controller 6 of the present embodiment controls the X-ray tube 3 so as for X-rays emitted from the X-ray tube 3 to have an energy width whose upper limit UL is around a K-shell absorption edge of K-shell absorption edges for elements forming the semiconductor film 16, the K-shell absorption edge corresponding to the characteristic X-ray whose energy influences the blur in the image. Here, the K-shell absorption edge corresponding to the characteristic X-ray whose energy influences the blur is hereinunder referred to as a “blur-influencing K-shell absorption edge” as appropriate. Specifically, the X-ray tube controller 6 controls the X-ray tube 3 so as for emitted X-rays to have an energy width whose upper limit UL is larger than a blur-influencing K-shell absorption edge of the K-shell absorption edges for elements forming the semiconductor film 16, and is equal to or less than a preset value corresponding to the blur-influencing K-shell absorption edge. See numerals RA1 and RA2.

Now description is made to the blur-influencing K-shell absorption edge. The blur-influencing K-shell absorption edge may contain all absorption edges equal to or more than 15 keV with the maximum K-shell absorption edge. The blur-influencing K-shell absorption edge may contain the minimum K-shell absorption edge. That is, the blur-influencing K-shell absorption edge contains the K-shell absorption edge(s) for an element or elements entirely or partially. For instance, with the semiconductor film 16 of CdTe, the K-shell characteristic X-rays of Cd and Te are each high, i.e., approximately 30 keV as noted above. In addition, the K-shell characteristic X-rays of approximately 30 keV each have an attenuation length of approximately 100 μm in CdTe, and thus is long. Accordingly, a large amount of electric charges are detected within a wide area, leading to the blur in the image. Consequently, the K-shell absorption edges for the elements Cd and Te are each a blur-influencing K-shell absorption edge. Determination of whether or not the K-shell absorption edge corresponds to the blur-influencing K-shell absorption edge is made, for example, from a relationship between the attenuation length of K-shell characteristic X-ray and a pixel pitch. The X-ray tube controller 6 performs control regarding the minimum K-shell absorption edge of the K-shell absorption edges whose energy is 15 keV or more and influences the blur. However, when numeric values of the K-shell absorption edge are close to each other like CdTe, another K-shell absorption edge other than the minimum K-shell absorption edge may be controlled.

Moreover, the preset value corresponding to the blur-influencing K-shell absorption edge is the sum of the blur-influencing K-shell absorption edge and a given value F. Here, a given value F is prepared in advance through experiments. In the present embodiment, the preset value corresponding to the blur-influencing K-shell absorption edge has been described as the sum of the blur-influencing K-shell absorption edge and +40%, for example. Here, the given value F is not limited to +40%.

FIG. 5A illustrates a relationship between the K-shell absorption edges for the semiconductor film of CdTe and emitted X-ray energy (X-rays spectrum) XS2. With the semiconductor film 16 of CdTe, both the K-shell absorption edges for Cd and Te are each the blur-influencing K-shell absorption edge.

The X-ray tube controller 6 controls the X-ray tube 3 so as to emit X-rays whose energy is set with reference to the blur-influencing K-shell absorption edge for either Cd or Te. For instance, with reference to approximately 27 keV as the K-shell absorption edge for Cd, the X-ray tube controller 6 controls the X-ray tube 3 so as to emit X-rays having an energy width whose upper limit UL is larger than approximately 27 keV and equal to or less than the sum of approximately 27 keV and +40% (e.g., approximately 37.8 keV). See numeral RA2. This achieves a smaller diagonally shaded area in FIG. 5A than the diagonally shaded area in FIG. 4. Consequently, the number of ejected K-shell characteristic X-rays by Cd decreases, and accordingly the number of ejected K-shell characteristic X-rays by Te decreased. An amount of electric charges generated from the K-shell characteristic X-ray also decreases with the decreased number of ejected K-shell characteristic X-rays. This achieves the suppressed blur in the image.

With the semiconductor film 16 composed of an element such as Si or Se, the blur-influencing K-shell absorption edge corresponds to the K-shell absorption edge for the element.

In addition, the following may be implemented. With the semiconductor film 16 composed of many different types of elements, the K-shell absorption edges other than the maximum K-shell absorption edge and smaller than the K-shell absorption edge for Cd are used as illustrated by the numeral E1 in FIG. 5A. Consequently, the K-shell characteristic X-ray emitted from the elements of the K-shell absorption edges other than the maximum K-shell absorption edge has energy lower than that for Cd. Smaller energy of the K-shell characteristic X-ray achieves a suppressed attenuation length of the K-shell characteristic X-rays. Accordingly, a less amount of electric charges from the K-shell characteristic X-ray is obtainable. In order to achieve the above, the semiconductor film 16 composed of ZnTe or InP, for example, is used.

In this modification, it is assumed that the blur-influencing K-shell absorption edge contains at least the maximum K-shell absorption edge. In addition, the K-shell absorption edge other than the blur-influencing K-shell absorption edges (containing the maximum K-shell absorption edge) contains at least the minimum K-shell absorption edge. Moreover, the K-shell absorption edge may be less than 15 keV.

The following may be implemented. Reference is made to FIG. 5B. The semiconductor film 16 is composed of many different types of elements. The K-shell absorption edge of the K-shell absorption edges for elements forming the semiconductor film 16 other than the maximum K-shell absorption edge is less than 15 keV. The maximum K-shell absorption edge is for elements having higher energy than that in an energy area to be contrasted. In order to achieve the above, the semiconductor film 16 is composed of, for example, TlBr. The K-shell absorption edges for two elements Tl and Br forming TlBr are approximately 85 keV and 12 keV, respectively. As noted above, the X-ray tube controller 6 controls the X-ray tube 3 so as to emit X-rays with an energy width whose upper limit UL is larger than approximately 85 keV and equal to or less than the sum of 85 keV and +40%. See the numeral RA1.

Accordingly, since the K-shell absorption edge for many different types of elements forming the semiconductor film 16 other than the maximum K-shell absorption edge is less than 15 keV, energy of the K-shell characteristic X-ray emitted from the elements containing the K-shell absorption edges other than the maximum K-shell absorption edge can be set lower than that of Cd. Moreover, since the maximum K-shell absorption edge is large, X-rays whose energy is higher than that of CdTe can be emitted. Even if X-rays having more energy than the maximum K-shell absorption edge are emitted, almost X-rays have energy less than the absorption edge as long as the energy are close to the maximum K-shell absorption edge. Consequently, influence of blur due to the K-shell characteristic X-ray depending on the maximum K-shell absorption edge can be suppressed.

In this modification, it is similarly assumed that the blur-influencing K-shell absorption edge contains at least the maximum K-shell absorption edge. In addition, the K-shell absorption edge other than the blur-influencing K-shell absorption edge (containing the maximum K-shell absorption edge) contains at least the minimum K-shell absorption edge. Moreover, the K-shell absorption edge may be less than 15 keV. Moreover, in the above description, the K-shell absorption edge other than the maximum K-shell absorption edge is less than 15 keV. Alternatively, the K-shell absorption edge other than the maximum K-shell absorption edge may be less than the absorption edge for Cd. Here, energy higher than energy in an area to be contrasted is several tens keV or more. Examples of the energy include energy higher than the K-shell absorption edge for Te.

The following describes basic operation of the X-ray apparatus 1 and action through control of the X-ray tube controller 6 depending on the semiconductor film 16. Firstly, description is made to the basic operation of the X-ray apparatus 1.

As illustrated in FIG. 1, the subject M is placed on the top board 2. An operator inputs necessary information via the input unit 10. The main controller 8 transmits setting data, such as tube voltage, corresponding to a material of the semiconductor film 16 to the X-ray tube controller 6 in association with the operator's input. The X-ray tube controller 6 controls the X-ray tube 3 to emit X-rays in accordance with the setting data. X-rays are emitted to the subject M on the top board 2. X-rays passing through the subject M enter into the semiconductor film 16 of the FPD 4.

In FIGS. 2 and 3, bias voltage Vh is applied to the common electrode 17. A potential difference between the common electrode 17 and the pixel electrodes 18 causes formation of an electric field within the semiconductor film 16. Consequently, electric charges generated in the semiconductor film 16 are shifted, and the electric charges are collected in the pixel electrodes 18 and are accumulated in the capacitors 21. The TFTs (thin-film transistors) actuate to cause the electric charges accumulated in the capacitors 21 to be read out to a data line 25 side. The gate driver circuit 26 transmits driving signals to the gate lines 24 beginning at the top, thereby actuating the TFTs 22 for the gate lines 24 individually.

If the TFT 22 actuates to be turned ON, the electric charges accumulated in the capacitor 21 are transmitted via the TFT 22 to the data line 25, through which the electric charges are transmitted to the charge-voltage converter group 27, the data line 25, and the multiplexer 28 in this order. The charge-voltage converter group 27 amplifies the electric charges and converts the electric charges into voltage signals. The multiplexer 28 selects and outputs one of the voltage signals. The A/D converter 29 converts the analog image to a digital image.

The image processor 7 performs necessary processing, such as contrast adjustment, to the image outputted from the FPD 4. The image processed by the image processor 7 is stored in the storing unit 9, and is displayed on the display unit 11.

The following describes action of X-ray emission by the X-ray tube controller 6.

FIG. 2 schematically illustrates generation of electric charges when photoelectric effect causes ejection of the K-shell characteristic X-ray (see the numeral XR2). When X-rays incident into the semiconductor film 16 causes a photoelectric effect in the semiconductor film 16, one photoelectron is ejected and electric charges (electron-hole pair) are generated until the photoelectron loses its kinetic energy. On the other hand, either a characteristic X-ray or Auger electron is ejected during transition of the electron losing its photoelectron from an excited state to a ground state.

With ejection of the characteristic X-ray, if the characteristic X-ray is absorbed in a pixel where X-rays enter, electric charges generated from an photoelectric effect of the characteristic X-ray are added to the electric charges generated from an photoelectric effect of incident X-rays, and thus are a part of events of the photoelectric effect by the incident X-rays. In contrast to this, when the characteristic X-ray is not absorbed in a pixel where X-rays enter and is ejected out of the pixel, i.e., an event referred to as an escape event occurs, electric charges occur within the pixel although the characteristic X-ray is absorbed out of an area of the original pixel where X-rays enter. Consequently, signals are generated. Moreover, with ejection of an Auger electron, the Auger electron generates electric charges (electron-hole pair) until the Auger electron loses its kinetic energy, which is similar to the case of photoelectron. The result is that energy of incident X-rays is entirely used for generating electron-hole pairs.

Reference is made to the above table 1. Table 1 indicates a list of energy of K-shell characteristic X-rays for CdTe and TlBr. The increased atomic number causes increased ejection probability of K-shell characteristic X-rays (yield of the K-shell characteristic X-rays). For CdTe, a photoelectric effect causes ejection of K-shell characteristic X-ray of approximately 30 keV at a probability of approximately 85%. If K-shell characteristic X-ray is ejected out of a pixel area where the photoelectric effect occurs due to incident X-rays (K-escape), electric charges may be ejected into another pixel area adjacent to the pixel area. The K-shell characteristic X-rays of 30 keV each have an attenuation length of approximately 100 μm in CdTe. Accordingly, a narrower pixel pitch causes a larger blur due to the K-shell characteristic X-rays.

For instance, if the subject M has a certain degree of thickness (thickness with a converted density) and X-rays are emitted with tube voltage of around 100 kV, TlBr is used as the semiconductor film 16. Usage of the tube voltage of around 100 kV or less achieves suppression of the blur due to K-escape like CdTe. Table 1 reveals that energy of K-shell characteristic X-rays ejected from TlBr (see, Kα and Kβ) is approximately 13 keV and 80 keV, respectively. For the tube voltage of 100 kV, X-rays from the X-ray tube 3 has energy of 100 keV with no interposing member such as a filter. Accordingly, X-rays as the K-shell absorption edge for Tl whose emission energy of 85 keV or more are present. However, a dose of X-rays whose energy is more than 85 keV is less than 10% of the total. Consequently, K-shell characteristic X-ray mainly ejected has energy of approximately 13 keV. The K-shell characteristic X-rays of approximately 13 keV each have a short attenuation length of around 20 μm in TlBr. Accordingly, the number K-escape events decreases, and an amount of electric charges to be generated is also small. This achieves the suppressed blur in the image.

<Simulation Result (1)>

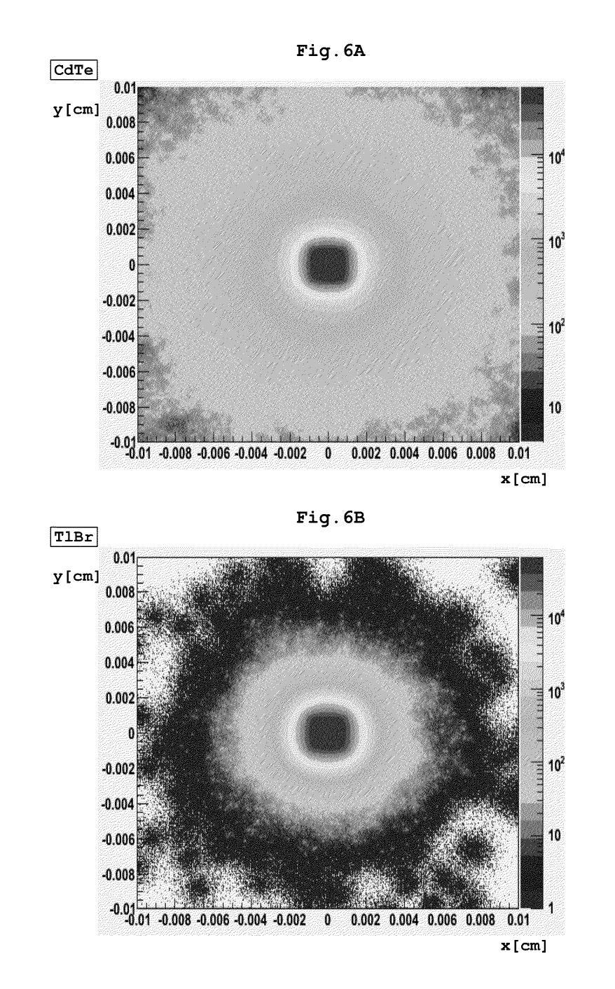

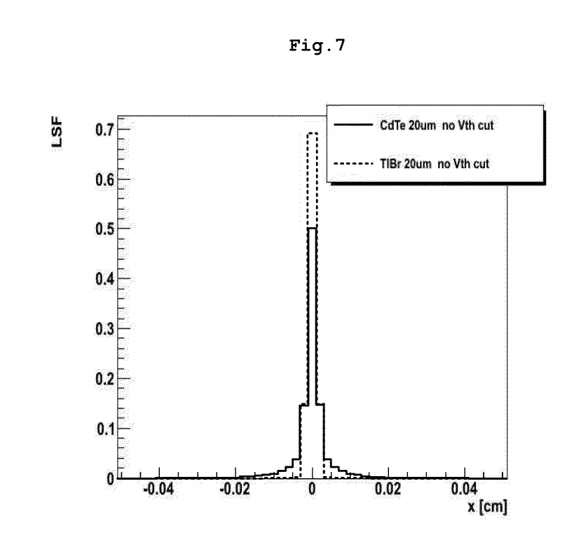

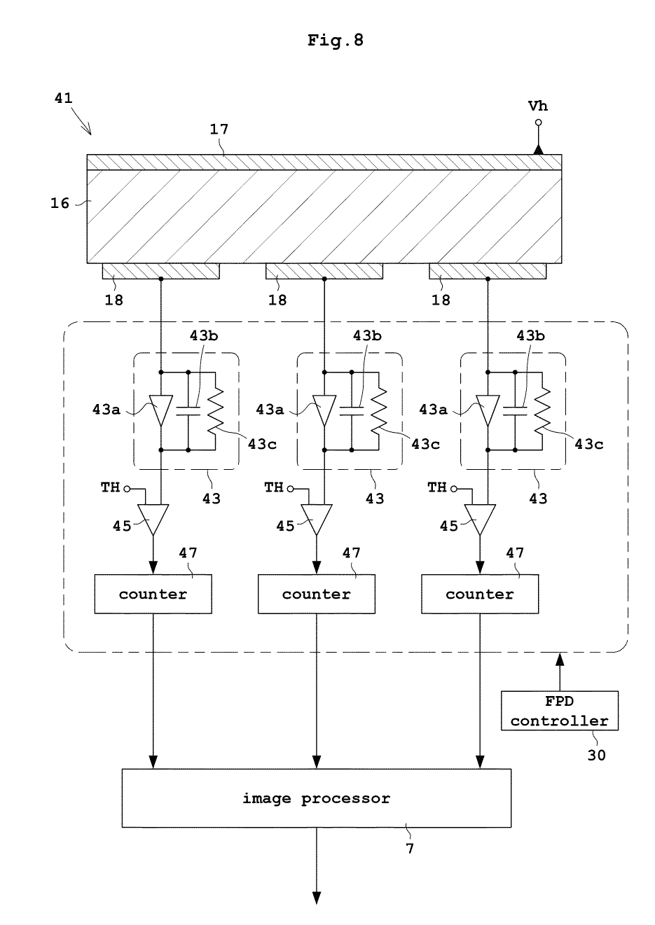

FIGS. 6A and 6B each illustrate a simulation result. FIG. 6A illustrates a simulation result with CdTe for the semiconductor film 16, and FIG. 6B illustrates a simulation result with TlBr for the semiconductor film 16. Through the simulations, a distribution of detected electric charges is observed upon uniform emission of one hundred thousand beams of X-rays with a spectrum corresponding to tube voltage of 100 kV are applied to a pixel of 20 μm sq. (20 μm by 20 μm). Here in the simulations, the conversion film has a thickness of 500 μm and bias voltage of 200V. FIGS. 6A and 6B reveals that the detected electric charges for TlBr spread less widely than the detected electric charges for CdTe, and thus the suppressed blur in the image derived from K-escape is obtainable. FIG. 7 illustrates a distribution of detected electric charges obtained through integration of FIGS. 6A and 8b in the vertical direction along a paper plane with all amounts of electric charges for the drawings of 1.0. It is revealed that TlBr spreads less widely than CdTe, and thus spreads abruptly.

With the present embodiment, the X-ray tube controller 6 controls the X-ray tube 3 so as for the X-rays emitted from the X-ray tube 3 to have an energy width whose upper limit UL is more than the blur-influencing K-shell absorption edge of the K-shell absorption edges for the elements forming the semiconductor film 16 and equal to or less than the preset value depending on the blur-influencing K-shell absorption edge of the K-shell absorption edges for the elements. Moreover, the X-ray tube controller 6 controls the X-ray tube 3 so as for the X-rays emitted from the X-ray tube 3 to have an energy width whose upper limit is more than the K-shell absorption edge for the element forming the semiconductor film 16 and equal to or less than the preset value depending on the blur-influencing K-shell absorption edge for the element. That is, emitted X-rays have an energy width whose upper limit is controlled depending on the K-shell absorption edge of the element forming the semiconductor film 16. Accordingly, the less number of ejected K-shell characteristic X-rays is obtainable than the case when the emitted X-rays have an energy width whose upper limit is more than the value preset depending on the blur-influencing K-shell absorption edge. Consequently, the suppressed blur in an image is obtainable, the blur occurring due to ejection of K-shell characteristic X-ray outside a pixel area where X-rays enter to introduce a photoelectric effect.

Moreover, the X-ray tube controller 6 controls the X-ray tube 3 so as for the X-rays to have an energy width whose upper limit UL is more than the blur-influencing K-shell absorption edge. This achieves emission of X-rays with higher energy.

The semiconductor film 16 of TlBr is composed of many different types of elements. The K-shell absorption edge for Br of the K-shell absorption edges for Tl and Br (elements) forming the semiconductor film 16 other than the K-shell absorption edge (blur-influencing K-shell absorption edge) for Tl is less than 15 keV. This achieves ejection of the K-shell characteristic X-ray with less energy, and thus a less amount of electric charges generated from the K-shell characteristic X-ray is obtainable. Accordingly, a less amount of electric charges is generated even when the K-shell characteristic X-ray reaches the pixel other than the pixel to which incident X-rays enter to introduce a photoelectric effect. This achieves the suppressed blur in the image. In addition, the K-shell absorption edge for Tl is larger than the K-shell absorption edge for Te. Accordingly, an upper limit of an energy width of emission X-rays is set with reference to the K-shell absorption edge of 15 keV or more. Accordingly, emission of X-rays with higher energy is obtainable.

Moreover, the pixel electrodes 18 each have a pitch of several tens μm or less. Accordingly, the suppressed blur in the image is obtainable with the pitches of the pixel electrodes 18 each equal to or less than several tens μm.

### EMBODIMENT 2

The following describes Embodiment 2 of the present invention with reference to drawings. FIG. 8 illustrates a flat panel X-ray detector (FPD) according to Embodiment 2. Here, the description common to that of Embodiment 1 is to be omitted.

The FPD 4 of Embodiment 1 adopts an integral reading system for a reading system. An FPD 41 in Embodiment 2 adopts a photon counting system. FIG. 8 illustrates a flat panel X-ray detector (FPD) according to Embodiment 2.

The FPD 41 of Embodiment 2 includes a charge-voltage converter 43 as a read-out circuit for processing electric charges collected in pixel electrodes, an output comparator 45, and a counter 47. The charge-voltage converter 43 converts the electric charges collected for every pixel by the pixel electrodes 18 into voltage signals. The comparator 45 outputs a photon detection signal indicating detection of one photon when the electric signal converted by the charge-voltage converter 43 has a value larger than a preset threshold TH. At the preset threshold TH, energy having values equal to or less than a value corresponding to energy of the K-shell characteristic X-ray is cut off. The counter 47 counts the number of photons for every pixel in accordance with the photon detection signal outputted from the comparator 45. The charge-voltage converter 43, the comparator 45, and the counter 47 are controlled by an FPD controller 30.

Similar to Embodiment 1, the pixel electrodes 18 are arranged for the pixels individually. The counter 47 corresponds to the collecting device in the present invention.

The charge-voltage converter 43 includes amplifiers 43a, capacitors 43b, and resistors 43c. The comparator 45 compares the voltage signal converted by the charge-voltage converter 43 with the preset threshold TH. If the voltage signal is higher than the preset threshold TH, a photon detection signal indicating detection of one photon is outputted. The voltage signal (pulse height value) corresponding energy of the K-shell characteristic X-ray whose value is equal to or less than the threshold TH is cut. In addition, the threshold TH is set for every pixel depending on uneven sensitivity and dark current. Moreover, the threshold TH is not necessarily set for every pixel, but may be equal among all the pixels. Moreover, like the charge-voltage converter 43 in FIG. 8, the amplifier 27a in FIG. 3 may include amplifiers 43a, capacitors 43b, and resistors 43c.

In FIG. 8, the comparator 45 and the counter 47 are provided for each of the pixel electrodes 18. Alternatively, the multiplexer as in FIG. 3 is provided on an output side of the charge-voltage converter 43 for every line in a Y-direction of pixels arranged in a two-dimensional matrix for reduction in number of comparators 45.

The following describes operation of the FPD 41 of the photon counting system. As in Embodiment 1, the X-ray tube controller 6 performs control depending on the semiconductor film 16 to the X-ray tube 3 to emit X-rays to the subject M on the top board 2. X-rays passing through the subject M enter into the semiconductor film 16 of the FPD 41.

In FIG. 8, bias voltage Vh is applied to the common electrode 17. A potential difference between the common electrode 17 and the pixel electrodes 18 causes formation of an electric field in the semiconductor film 16. The electric field causes shift of the electric charges generated in the semiconductor film 16. The shifted electric charges are collected in the pixel electrodes 18. The charge-voltage converter 43 amplifies the collected electric charges for each of the pixel electrodes 18, and converts the electric charges into voltage signals. The comparator 45 outputs the photon detection signal indicating detection of one photon when the electric signal converted by the charge-voltage converter 43 is larger than the preset threshold TH. Here, at the preset threshold TH, energy having values equal to or less than a value corresponding to energy of the K-shell characteristic X-ray is cut off. When the electric signal converted by the charge-voltage converter 43 is smaller than the threshold TH, no photon detection signal is outputted. However, a non-counted signal indicating no photon detection may be outputted.

The counter 47 counts the number of photons for every pixel in accordance with the photon detection signal outputted from the comparator 45. After the counter 47 counts a sufficient number of photons entirely in the two-dimensional direction, data counted for every pixel is outputted as an image.

The image processor 7 performs necessary processing, such as contrast adjustment, to the image outputted from the FPD 41. The image processed by the image processor 7 is stored in the storing unit 9, and is displayed on the display unit 11.

As illustrated in FIGS. 6A and 7, a distribution of the number of electric charges (voltage signal) is relatively gentle in a conventional blurred image. The number of detected photons significantly decreases depending on the threshold TH of the comparator 45. Consequently, imaging with soft X-rays having low emission X-ray energy is not performable. Accordingly, in Embodiment 1, the X-ray tube controller 6 performs control depending on the semiconductor film 16 to suppress the blur in the image. Consequently, a distribution of the number of electric charges (voltage signal) with a wide gentle area is changed into that with a narrow abrupt area. In addition, the threshold is discriminated and counted in the present embodiment. Consequently, reduction in number of detected photons can be suppressed.

<Simulation Result (2)>

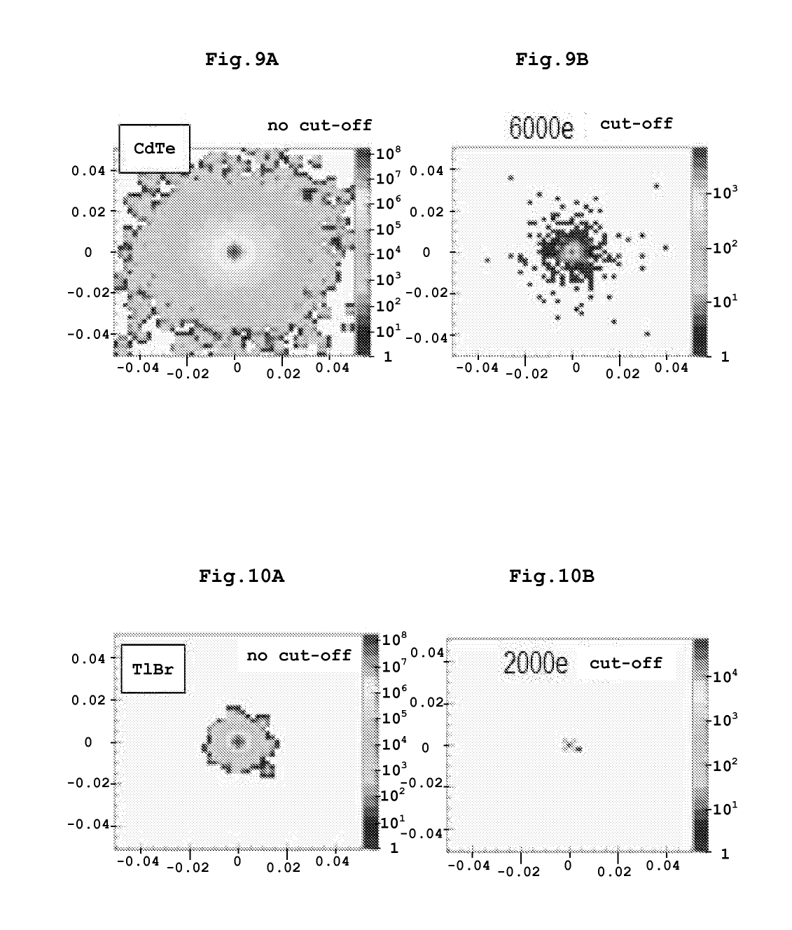

FIGS. 9A, 9B, 10A and 10B each illustrate a simulation result. These drawing s differ from the above FIG. 6A in that one bin (section) of each two-dimensional histogram corresponds to a pixel of 20 μm square. Moreover, similar to the above FIG. 6A, the simulation is conducted under conditions of tube voltage of 100 keV, a thickness of the semiconductor film 16 of 500 μm, and bias voltage of 200V.

FIGS. 9A and 9B each illustrate the semiconductor film 16 of CdTe. FIGS. 10A and 10B each illustrate the semiconductor film 16 of TlBr. In addition, FIG. 9A indicates the presence or absence of threshold discrimination. Specifically, FIGS. 9A and 10A each illustrate an electric charge distribution with no threshold discrimination using the integral reading system of Embodiment 1 as the reading system. In contrast to this, FIGS. 9B and 10B each illustrate a detected electric charge distribution with threshold discrimination using the photon counting system as the reading system.

FIGS. 9B and 10B each illustrate the result with the number of electrons (the number of electric charges) corresponding to the K-shell characteristic X-ray of the semiconductor film 16 as the threshold TH. In FIG. 9B for CdTe, a value “6000e” is set as the threshold TH. In FIG. 10B for TlBr, a value “2000e” is set as the threshold TH. In FIG. 8, the charge-voltage converter 43 converts the electric charge into the voltage signal. The comparator 45 uses a value obtained from the voltage signal converted from an electric charge of “6000e”, for example, as the threshold TH for discrimination.

The threshold discrimination causes less-wide spread of the electric charge distributions of CdTe and TlBr, respectively, in FIGS. 9B and 10B than that in FIGS. 9A and 10A. For CdTe, when the threshold TH is set by the number of electric charges of around 30 keV, the electric charge distribution is relatively gentle. Accordingly, approximately nine-tenths of X-rays up to 60 keV are not detectable. An electric charge distribution for TlBr spreads less widely also with the integral reading system. Accordingly, similar to the distribution for CdTe, significant reduction in number of detected photon can be suppressed upon the threshold discrimination.

The FPD 41 of the present embodiment includes a charge-voltage converter 43 as a read-out circuit for processing electric charges collected in pixel electrodes, an output comparator 45, and a counter 47. The charge-voltage converter 43 converts the electric charge collected for each of the pixel electrodes 18 into a voltage signal. The comparator 45 outputs a photon detection signal, indicating detection of one photon, when the voltage signal converted by the charge-voltage converter 43 is higher than a preset threshold TH. At the threshold TH, a voltage signal equal to or less than the energy of the K-shell characteristic X-ray is cut off. The counter 47 counts the number of photons for every pixel in accordance with the photon detection signal.

The voltage signal corresponding to the energy of the K-shell characteristic X-ray equal to or less than the threshold TH is cut off. If the voltage signal converted with the charge-voltage converter 43 is higher than the threshold TH, the comparator 45 outputs the photon detection signal indicating detection of one photon. Accordingly, suppressed detection of photons is obtainable within pixels other than pixels to which X-rays enter. This leads to a suppressed blur in the image. Moreover, as noted above, control of the X-ray tube controller 6 causes suppressed ejection of K-shell characteristic X-rays. This causes an abrupt distribution of the voltage signals obtained from the incident X-rays. Consequently, reduction in number of detected photons can be suppressed. The reduction occurs when the comparator 45 discriminates electric signals in the pixels to which emitted X-rays enter as no photon detection using the preset threshold. That is, suppression in wasted dose of X-rays is obtainable.

The present invention is not limited to the foregoing examples, but may be modified as follows.

(1) As illustrated in FIGS. 5A and 5B in the embodiments mentioned above, the X-ray tube controller 6 controls the X-ray tube 3 so as for emitted X-rays from the X-ray tube 3 to have an energy width whose upper limit UL is larger than a blur-influencing K-shell absorption edge of the K-shell absorption edges for the elements forming the semiconductor film 16, and is equal to or less than a preset value corresponding to the blur-influencing K-shell absorption edge. See numerals RA1 and RA2. However, this is not limitative. For instance, the X-ray tube controller 6 may control the X-ray tube 3 so as for emitted X-rays to have an energy width whose upper limit UL is larger than the minimum K-shell absorption edge of the K-shell absorption edges for the elements forming the semiconductor film 16. See numeral RA3.

That is, X-rays with higher energy is usable when the emitted X-rays has an energy width whose upper limit UL is more than the maximum K-shell absorption edge of the blur-influencing K-shell absorption edges. On the other hand, the upper limit UL may be equal to or less than the maximum K-shell absorption edge. The upper limit UL of the energy width of the emitted X-rays adjacent to the minimum K-shell absorption edge allows the suppressed number of ejected K-shell characteristic X-rays at the minimum K-shell absorption edge. Moreover, the upper limit UL of the energy width of the emitted X-rays adjacent to the maximum K-shell absorption edge allows emission of X-rays with higher energy. It should be noted that the above is focused on the area larger than the minimum K-shell absorption edge. This is because no K-shell characteristic X-ray is ejected at an area corresponding to the minimum K-shell absorption edge or less.

For instance, if the subject M has a small thickness (thickness with a converted density) and an area with soft X-rays of 30 keV or less is used, CdTe is used for the semiconductor film 16. Since the K-shell absorption edge for CdTe is approximately 30 keV, the blur in the image can be suppressed even if the K-shell characteristic X-ray is ejected.

In this modification, the minimum K-shell absorption edge may be equal to the blur-influencing K-shell absorption edge. In such a case, the upper limit UL of the X-ray energy width falls within the area in FIG. 5A denoted by the numeral RA2. In addition, the lower end of the upper limit UL of the emitted X-ray energy is set with reference to the minimum K-shell absorption edge. Alternatively, the lower end may be set with reference to the K-shell absorption edge other than the minimum K-shell absorption edge.

(2) The present embodiments and the modification (1) mentioned above each describe the blur-influencing K-shell absorption edge as the K-shell absorption edge corresponding to the characteristic X-ray whose energy influences the blur. In addition, the blur-influencing K-shell absorption edge may be 15 keV or more. That is, the K-shell absorption edge of 15 keV or more among the K-shell absorption edges for the elements forming the semiconductor film 16 is used as the blur-influencing K-shell absorption edge. This leads to control by the X-ray tube controller 6 with reference to the blur-influencing K-shell absorption edge as the K-shell absorption edge of 15 keV or more. On the other hand, if the blur-influencing K-shell absorption edge is less than 15 keV, the ejected K-shell characteristic X-ray has a small attenuation length. Accordingly, the K-shell characteristic X-ray spreads less widely. In addition, the ejected K-shell characteristic X-ray has low energy, causing a less amount of electric charges generated from the K-shell characteristic X-ray. For instance, the K-shell characteristic X-ray ejected from Br of TlBr is around 13 keV and has an attenuation length of around 20 μm. In addition, the K-shell characteristic X-ray of around 13 keV generates a less amount of electric charges.

The K-shell absorption edge less than 15 keV does not correspond to the blur-influencing K-shell absorption edge. Accordingly, the K-shell absorption edge may not be considered upon control of X-rays for influence of no blur. That is, the X-ray tube controller 6 controls the X-ray tube 3 so as for X-rays emitted from the X-ray tube 3 to have an energy width whose upper limit UL is equal to or more than 15 keV and less than the preset value depending on the blur-influencing K-shell absorption edge of the K-shell absorption edges for the elements.

(3) In the present embodiments and each modification mentioned above, the semiconductor film 16 is composed of many different types of elements. The K-shell absorption edge, other than the blur-influencing K-shell absorption edge, of the K-shell absorption edges for the elements forming the semiconductor film 16 may have energy whose attenuation length of the ejected K-shell characteristic X-ray is less than twice a pitch P of the pixel electrode 18. Accordingly, the ejected K-shell characteristic X-ray falls within an area smaller than twice the pitch P of the pixel electrode 18. This achieves the suppressed blur in the image. For instance, the K-shell characteristic X-ray ejected from Br of TlBr is around 13 keV, and has an attenuation length of around 20 μm. If the pitch P of the pixel electrode 18 is less than 50 μm, the blur in the image falls within a 2-pixel pitch.

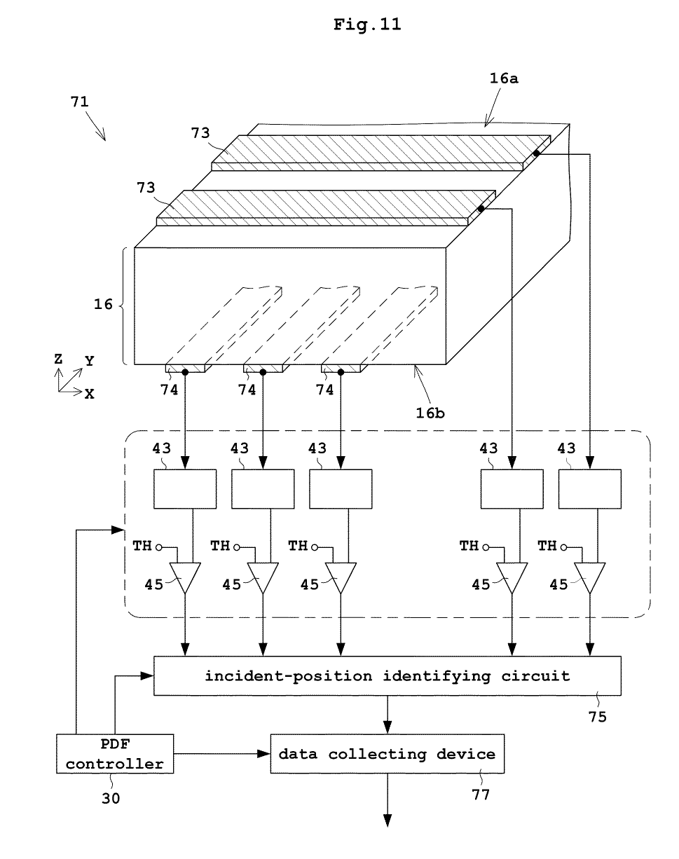

(4) The present embodiments and each modification mentioned above each describe the pixel electrodes 18 as one example of a plurality of collecting electrodes arranged on the semiconductor film 16 for collecting the electric charges converted with the semiconductor film 16. Alternatively, the collecting electrodes may be strip electrodes 73 and 74 of an FPD 71 in FIG. 11.

The following describes the FPD 71. The longitudinal strip electrodes 73 in an X-direction are arranged on the semiconductor film 16 on an X-ray incident side 16a. The longitudinal strip electrodes 74 in a Y-direction are arranged on the semiconductor film 16 on an opposite side 16b of the X-ray incident side. The Y-direction intersects the X-direction. The strip electrodes 73 and 74 are arranged almost orthogonal to each other. The strip electrodes 73 and the strip electrodes 74 are arranged individually in parallel.

Incidence of X-rays into the semiconductor film 16 causes generation of electric charges (positive hole and electron). For instance, bias voltage is applied to the strip electrode 73. A potential difference between the strip electrode 73 and 74 forms an electric field in the semiconductor film 16. Accordingly, the positive hole and electron generated in the semiconductor film 16 travels in opposite directions to be collected in the strip electrodes 73 and 74, respectively.

The collected positive hole and electron are converted into voltage signals with the charge-voltage converter 43, and are discriminated based on the threshold TH preset by the comparator 45. If the voltage signals are higher than the threshold TH, photon detection signals are outputted. Then an incident-position identifying circuit 75 determines an incident position of X-rays in accordance with the photon detection signals on sides adjacent to the strip electrode 73 and the strip electrode 74. A data collecting device 77 collects data on an X-ray incident position (pixel) determined by the incident-position identifying circuit 75 and the number of incident X-rays (the number of detected photons), and outputs an X-ray image.

Similar to the case with the strip electrodes 73 and 74 as the collecting electrodes, the number of detected photons significantly decreases in the blurred image depending on the threshold TH of the comparator 45. The incident-position identifying circuit 75 determines the incident position of X-rays in accordance with the photon detection signals on the sides adjacent to the strip electrode 73 and the strip electrode 74. Accordingly, reduction in number of detected photons causes a less number of determinable incident positions of X-rays. However, in Embodiment 1, control of the X-ray tube controller 6 depending on the semiconductor film 16 causes the suppressed blur in the image. Accordingly, a distribution of the number of electric charges (voltage signal) with a wide gentle area is changed into that with a narrow abrupt area. In addition, the threshold discrimination and counting is performed, achieving the significantly suppressed number of detected photons. Here, the incident-position identifying circuit 75 and the data collecting device 77 correspond to the collecting device in the present invention.

(5) The present embodiments and each modification mentioned above describe the semiconductor film 16 sensitive to incident X-rays to generate electric charges as the conversion film. In other words, the semiconductor film 16 is of a direct conversion type. Alternatively, the conversion film may be of an indirect conversion film. As noted above, for the indirect conversion film type, a reaction position of X-rays on the scintillator differs from a position where a photodiode catches X-rays. However, the semiconductor film 16 of the indirect conversion type achieves the suppressed blur in the image as long as the above difference is not considered.

The indirect conversion film is formed by scintillators converting X-rays into another type of light, and light receiving elements, such as photodiodes, converting the other type of light converted with the scintillators into electric charges. A photoelectric effect occurs between the scintillators and the light receiving elements. FIG. 2, the semiconductor film 16 may be replaced by the scintillators, and the pixel electrodes 18 may be replaced by the photodiodes and pixel electrodes. The pixel electrode in the modification corresponds to the electrode collecting the electric charges converted with the photodiode, and thus may be a part of the photodiode.

(6) In the present embodiments and each modification mentioned above, the X-ray apparatus 1 includes a plurality of FPDs 4 (or 41 and 71). The FPDs 4 each include different semiconductor films 16 (e.g., CdTe and TlBr). The X-ray tube controller 6 controls the X-ray tube 3 depending on one selected FPD 4. Such is adoptable. Either the FPD 4 with the semiconductor film 16 of CdTe or the FPD 4 with the semiconductor film 16 of TlBr is selected depending on inspections. The X-ray tube controller 6 controls the X-ray tube 3 depending on the semiconductor film 16 of the FPD 4. This achieves the suppressed blur in the image even in X-rays having energy different depending on the inspections are emitted.

The present invention may be embodied in other specific forms without departing from the spirit or essential attributes thereof and, accordingly, reference should be made to the appended claims, rather than to the foregoing specification, as indicating the scope of the invention.

### REFERENCE SIGNS LIST

1 . . . X-ray apparatus
3 . . . X-ray tube
4, 41, 71 . . . flat panel X-ray detector (FPD)
6 . . . X-ray tube controller
8 . . . main controller
16 . . . semiconductor film
18 . . . pixel electrode
27 . . . charge-voltage converter group
43 . . . charge-voltage converter
45 . . . comparator
47 . . . counter
51 . . . read-out substrate
51a . . . wiring
53 . . . IC chip
53a . . . wiring
55, 61 . . . bump
59 . . . through-silicon-via electrode (TSV)
73, 74 . . . strip electrode
75 . . . incident-position identifying circuit
77 . . . data collecting device
UL . . . upper limit of energy width of emitted X-rays

What is claimed is:

1. An X-ray apparatus conducting X-ray radiography, comprising:
an X-ray tube emitting X-rays to a subject;
an X-ray detector detecting X-rays passing through the subject; and
an X-ray tube controller controlling the X-ray tube,
the X-ray detector including a conversion film and collecting electrodes, the conversion film being composed of many different types of elements and converting incident X-rays into electric charges, the collecting electrodes being provided on at least one face of the conversion film, and each collecting the electric charges converted with the conversion film, and
the X-ray tube controller controlling the X-ray tube so as for the X-rays emitted from the X-ray tube to have an energy width whose upper limit is more than the minimum K-shell absorption edge of K-shell absorption edges for the elements forming the conversion film and is equal to or less than a preset value depending on a K-shell absorption edge of the K-shell absorption edges for the elements corresponding to a characteristic X-ray whose energy influences a blur.

2. An X-ray apparatus conducting X-ray radiography, comprising:
an X-ray tube emitting X-rays to a subject;
an X-ray detector detecting X-rays passing through the subject; and
an X-ray tube controller controlling the X-ray tube,
the X-ray detector including a conversion film and collecting electrodes,
the conversion film being composed of one type of element and converting incident X-rays into electric charges, the collecting electrodes being provided on at least one face of the conversion film, and each collecting the electric charges converted with the conversion film, and
the X-ray tube controller controlling the X-ray tube so as for the X-rays emitted from the X-ray tube to have an energy width whose upper limit is more than a K-shell absorption edge for the element and is equal to or less than a preset value depending on the K-shell absorption edge for the element corresponding to the characteristic X-ray whose energy influences the blur.

3. The X-ray apparatus according to claim 1, wherein
the X-ray detector includes a charge-voltage converter, a comparator, and a collecting device,
the charge-voltage converter converting the electric charge collected in the collecting electrodes individually into a voltage signal,
the comparator outputting a photon detection signal, indicating detection of one photon, if the voltage signal converted with the charge-voltage converter is higher than a preset threshold, at the preset threshold a voltage signal equal to or less than energy corresponding to a K-shell characteristic X-ray being cut off, and
the collecting device counting the number of photons for each of pixels in accordance with the photon detection signal.

4. The X-ray apparatus according to claim 1, wherein
the K-shell absorption edge corresponding to the characteristic X-ray whose energy influences the blur is 15 keV or more.

5. The X-ray apparatus according to claim 1, wherein
a K-shell absorption edge among K-shell absorption edges for the elements forming the conversion film, other than the K-shell absorption edge corresponding to the characteristic X-ray whose energy influences the blur, is lower than a K-shell absorption edge for Cd.

6. The X-ray apparatus according to claim 5, wherein
the K-shell absorption edge corresponding to the characteristic X-ray whose energy influences the blur is higher than a K-shell absorption edge for Te

7. The X-ray apparatus according to claim 1, wherein
a K-shell absorption edge among K-shell absorption edges other than the K-shell absorption edge corresponding to the characteristic X-ray whose energy influences the blur has energy with which the K-shell characteristic X-ray to be ejected has an attenuation length smaller than twice a pitch of the collecting electrode.

8. The X-ray apparatus according to claim 1, wherein
the X-ray tube controller controls the X-ray tube so as for the upper limit of the energy width to be more than the K-shell absorption edge corresponding to the characteristic X-ray whose energy influences the blur.

9. The X-ray apparatus according to claim 1, wherein
the collecting electrodes each have a pitch equal to or less than several tens μm.
